(19) United States
(12) Patent Application Publication
(10) Pub. No.: US 2024/0033693 A1
(43) Pub. Date: Feb. 1, 2024

(54) APPARATUS AND METHOD FOR PREPARING A DIALYSIS SOLUTION

(71) Applicant: VIVONIC GMBH, Sailauf (DE)

(72) Inventors: Patrick BESSLER, Erlenbach (DE); Stefan EBERLEIN, Höchberg (DE); Andreas HEMM, Alzenau (DE)

(73) Assignee: VIVONIC GMBH, Sailauf (DE)

(21) Appl. No.: 18/256,097

(22) PCT Filed: Dec. 8, 2021
(86) PCT No.: PCT/EP2021/084767

§ 371 Date: Jun. 6, 2023

(30) Foreign Application Priority Data
Dec. 8, 2020 (DE) 10 2020 132 659.9

(51) Int. Cl. B01F 21/00; B01F 25/312; B01F 25/53; B01F 21/20; B01F 35/221; B01F 35/213; A61M 1/16

(52) U.S. Cl. CPC B01F 21/30; B01F 25/3121; B01F 25/53; B01F 21/221; B01F 21/503; B01F 35/221; B01F 35/213; A61M 1/1656; B01F 2101/22

(57) ABSTRACT

The present invention relates to a device for preparing a dialysis concentrate or another fluid, wherein the device includes an inflow for the raw material for the dissolving of or mixing with a raw material located in a container that can be dissolved in the solvent or can be mixed with it and a container having a raw material to be dissolved by means of the solvent or miscible with the solvent, wherein the device has connection means for connecting to the container, characterized in that the device has a mixing unit that is self-regulating and a mixing line and a suction unit arranged in the mixing line and a mixing chamber, with the suction unit being able to be brought into fluid communication with the inflow for the solvent and, by means of a suction line, with the container connectable to the device, and with a return line downstream of the mixing chamber leading back to the container connectable to the device.

The present invention relates to a device for preparing a dialysis concentrate or another fluid, wherein the device includes an inflow for a solvent for dissolving or for mixing with a raw material located in a container, wherein the device has connection means for connecting to the container, and wherein the raw material is soluble in or mixable with the solvent.

The present invention further relates to an arrangement comprising a device in accordance with the invention having a container that is connected thereto and that contains the raw material, and to a method of preparing a dialysis concentrate or of another fluid, and to a blood treatment device having an arrangement in accordance with the invention.

The present invention further relates to the use of the device and arrangement in accordance with the invention for preparing a dialysis concentrate or for preparing another fluid, in particular a cleaning, sterilizing, or disinfecting fluid that Is in particular used for blood treatment devices, preferably for dialysis machines.

It is known from the prior art to supply dialysis machines with dialysis solutions or with dialysis concentrate via different paths.

The dialysis machines are frequently supplied with dialysis concentrate from a ring line system by means of a central supply unit. This is associated with the disadvantage of comparatively high investment costs for the ring line system and for the central supply system. A further disadvantage comprises the limited flexibility with respect to the composition of the dialysis concentrate since different dialysis concentrates can only be provided via the ring line to a very limited degree. If additional dialysis concentrates are required, this can only take place via other supply manners.

A further known procedure comprises supplying the dialysis machines with dialysis concentrate via canisters. The completed dialysis concentrate is in the canisters. This procedure is very flexible in comparison with the supply via the ring line, but is associated with the disadvantage of a high labor and time effort since every dialysis machine has to be individually equipped with a canister on every new treatment. In addition there is a disadvantage in that large storage areas are required for the storing of the canisters and the canisters have to be transported to the stores and from the stores to the dialysis machines, which is time consuming and bad for the environment since a large amount of water (as the solvent in the canisters) is transported that would per se also be available in the dialysis centers. It is finally disadvantageous that the canisters have to be disposed of after their single use, which is associated with a corresponding accumulation of plastic waste.

A further known procedure comprises completed dialysis solutions being used for the treatment, e.g. by means of peritoneal dialysis, that are produced centrally and are transported in a similar manner to the aforesaid canisters, but in bags, as a completed solution and are thus available for treatment locally.

A concentrate bag containing a raw material is known from the prior art in accordance with WO 99/06083 A1. The preparation of the dialysis concentrate takes place by adding water in the concentrate bag itself and by the dissolving of the raw material in the bag.

A device for preparing a dialysis concentrate in accordance with the preamble of claim 1 is known from DE 10 2017 127 637 A1. DE 10 2017 127 637 A1 discloses a fluid system having a main circuit and a branch section connected thereto for preparing dialysis concentrate. A mixing chamber and a container for receiving the concentrate, and a pump that pumps the fluid in the circuit in the main circuit are located in the main circuit. A container containing a raw material that is supplied to the main circuit is located in the branch section.

It is the underlying object of the present invention to provide a method and a device for preparing dialysis concentrate or also other solutions, wherein the investment costs and the time effort are small and there is simultaneously a high flexibility with respect to the solution to be prepared.

This object is achieved by a device having the features of claim 1, by an arrangement having the features of claim 9, by a blood treatment device having the features of claim 10, by a method having the features of claim 12, and by the use of a device in accordance with claim 17.

Provision is made in accordance with the invention that the device has a mixing unit that is self-regulating and comprises a mixing line and a suction unit arranged in the mixing line and a mixing chamber, wherein the suction unit can be brought into fluid communication with the inflow for the solvent and with the container by means of a suction line, and wherein a return line downstream of the mixing chamber leads back to the container connectable to the device.

With a container connected to the device, the raw material is sucked out of the container by the suction unit and is mixed with the water flowing in through the inflow or with the other solvent. This mixture then reaches the mixing chamber in which the mixing of the raw material with the solvent takes place. The solution prepared in this manner reaches the container via the return line.

Deviating from DE 10 2017 127 637 A1, the solution prepared in this manner is not supplied to a collection container, but rather to the container which the raw material was previously located.

The term “mixing” is also to be understood within the framework of the present invention as a dissolving procedure that is present when the raw material is solid or solid-liquid. The term “solution” can equally also relate to a purely mixing procedure that is present when the raw material is liquid.

The mixing process in the mixing chamber is self-regulating by the combination of the embodiment of the suction unit and the mixing chamber. A retention element is arranged in the mixing or fine mixing chamber and can e.g. be formed as a filter element. If undissolved raw material accumulates at this filter element, the pressure loss in the system rises and the throughflow through the filter element is reduced or is fully prevented. The flow rate via the suction unit and thus a further suction of further raw material from the container is thereby minimized. Even with a small flow, the undissolved raw material is flowed over by fresh solvent and the dissolving is thus optimized. If the raw material has dissolved sufficiently in the fine mixing chamber, the pressure loss falls and the flow rate of the solvent through the suction unit increases, which promotes a further suction of raw material through the suction unit into the fine mixing chamber. We also refer with respect to the carrying out of such a self-regulation to DE 10 2017 127 637 A1 whose disclosure content belongs to the subject matter of the present invention to this extent.

The mixing chamber can be a filter chamber, preferably a fine mixing filter chamber.

A batch preparation of a fluid concentrate, in particular of an acidic fluid concentrate, can be achieved by means of the present device.

The raw material present in the container, i.e. the raw concentrate, can have any desired aggregate state. The raw material can generally be solid, liquid, granular, powdery, slurry-like, or be present in another form.

The container is preferably disposable.

The container can be designed as a bag or also as a vessel having rigid walls, e.g. as a cartridge.

The container can be directly connected to a blood treatment device, preferably to a dialysis machine, and can thus have corresponding connection means that cooperate with correction means of the blood treatment device so that the container, preferably a bag, filled with dialysis concentrate can be connected to the blood treatment device.

In contrast to a procedure in which the raw material is dissolved in the raw material container, the mixing or dissolving takes place in accordance with the invention completely or substantially in the mixing chamber.

The container is connected via the suction line and via the return line to the blood treatment device or to another device for preparing the dialysis concentrate, etc. The suction line serves the removal of the raw material from the container and the return line serves the return transport of the completed concentrate into the container that is then present as a bag or the like filled with dialysis concentrate and can then be used for the preparation of the ready-to-use dialysis solution. When the container is connected, the opening of the suction line is preferably arranged in the proximity of the base of the container where the undissolved concentrate or solid, i.e. the undissolved raw material, accumulates.

The suction unit can, for example, be arranged in the inflow or in the mixing line or between them.

It can, for example, be formed as a Venturi nozzle whose partial vacuum is generated by the throughflow with solvent. If the inward flow of solvent is started, raw material or pre-concentrate is sucked from the container into the mixing line at the device side by the suction unit. The mixing chamber that—as stated above—sets the suction speed from the container in a self-regulating manner is located in said mixing line. In the ideal case, a completed concentrate can be prepared from the raw material by this regulation in a single passage, i.e. without a circuit flow of the raw material.

In accordance with the invention, a specific volume of solvent is preferably used that is fixed by the amount of electrolytes in the container. A batch having the desired volume and the desired composition can be prepared in this manner.

A valve is preferably arranged in the mixing line or in the inflow upstream of the suction unit.

A valve can furthermore be arranged in the mixing line downstream of the mixing chamber.

The device preferably does not have any mixing tank except for the mixing chamber and no other collection container for the prepared solution except for the container.

Provision is made in an embodiment that a drainage line for draining the completed dialysis concentrate or the other prepared fluid is provided, with the drainage line being fluidically connected to the container or to the suction line or to the mixing chamber.

If the total raw material has not yet been dissolved after the metering in of a specific solvent amount, a mixing circuit can be activated through which the concentrate circulates for so long until the undissolved substances still present in the mixing chamber have completely dissolved. This is in particular possible when the total raw material has already been sucked into the mixing chamber. This is one variant. However, the total raw material from the container does not have to be located in the mixing chamber so that the mixing circuit is activated since the mixing circuit or the fluid located therein circulates via the suction unit and can thus continue to suck in raw material from the container. An activation of the mixing circuit is thus also conceivable and covered by the invention if raw material is still located in the container.

The mixing circuit flow presents a flow via the suction unit so that raw material can also be sucked in without additional solvent having to be introduced into the mixing line.

A circuit line can thus be provided that is arranged in parallel with the suction unit and/or the mixing chamber and that opens into the mixing line upstream of the suction unit or of the mixing chamber and downstream of the mixing chamber.

A pump and preferably at least one valve can be arranged in the circuit line.

A sensor can be provided that is configured to detect a parameter value of the fluid (and/or its change over time) such as a concentration or conductivity in or downstream of the mixing chamber characteristic for the progress of the dissolving of the raw material, wherein a control unit in present that is connected to the sensor and that is configured to open or to close the circuit line in dependence on the parameter value. If insufficient solution is detected, the circuit line is opened so that the concentrate circulates for so long until a sufficient or complete dissolving has been achieved.

As stated above, the suction unit can be a Venturi nozzle.

The present invention further relates to a blood treatment device, in particular to a dialysis machine such as a hemodialysis machine or a peritoneal dialysis machine, having a device for preparing a dialysis concentrate or another fluid in accordance with the invention.

The blood treatment device preferably has a water inflow line in which water, in particular RO water, flows on the online preparation of ready-to-use-dialysis solution. A concentrate line that is in fluid communication with the container filled with liquid dialysis concentrate branches off from this inflow line. A concentrate pump can be arranged in the concentrate line for its conveying. A means for the measured conveying of water can also be located in the water inflow line so that a specific and desired dilution of the dialysis concentrate or the desired mixing ratio results. A mixing unit such as a mixing chamber can be provided downstream of the opening of the concentrate line into the water inflow line, in which mixing unit the dialysis concentrate is mixed with the water. It is generally conceivable and covered by the invention that a plurality of concentrate lines open into the water inflow line so that the ready-to-use dialysis solution can be prepared from a plurality of dialysis concentrates such as an acidic and a basic concentrate and water.

The subject of the disclosure is the device and/or the blood treatment device and/or the method and/or the use with and without a contained connected to the device or to the blood treatment device.

The present invention further relates to a method of preparing a dialysis concentrate or another fluid, wherein the method is carried out using a device or arrangement in accordance with the invention.

The preparation of the dialysis concentrate or the other fluid is preferably carried out without a circuit flow of the raw material via the container being produced. The solution itself moves back into the container after flowing through the mixing chamber and can be sucked from there back into the mixing line by means of the suction unit so that a circuit flow is possible via the container with respect to the solution and is covered by the invention.

The circuit line is flowed through when the solution of the raw material in the solvent is still not complete after metering in a specific amount of solvent.

If still undissolved raw material has remained in the container, some of the fluid or the total fluid can be branched off from the mixing line by a pump and can be conducted back through the suction unit in an embodiment. Where present, the remainder of the fluid can be conducted back into the container. Undissolved concentrate is then sucked from the container into the mixing line by the flow via the suction unit.

If a branching of a partial flow back into the container takes place, undissolved raw material can simultaneously be drawn from the container into the mixing line and a circulation in the container can be established by the container itself without the total volume of the concentrate having to be conducted in the mixing circuit.

In a conceivable embodiment, a first partial flow from the main line is branched off from the mixing line downstream of the suction unit and is again conducted via the suction unit and optionally the mixing chamber to convey still undissolved substances from the container. The remaining second partial flow is supplied to the container from the mixing line.

In a further embodiment, a degassing of the dialysis concentrate or of the other fluid in the mixing chamber takes place. A separate degassing device can be dispensed with in this case.

The present invention further relates to the use of a device in accordance with the invention for preparing a dialysis concentrate or for preparing another fluid, in particular a cleaning, sterilizing, or disinfecting fluid that Is in particular used for blood treatment devices, preferably for dialysis machines.

It is pointed out at this point that the terms “a” and “one” do not necessarily refer to exactly one of the elements, even though this represents a possible embodiment, but can also designate a plurality of elements. The use of the plural equally also includes the presence of the element in question in the singular and, conversely, the singular also includes a plurality of the elements in question.

Further advantages and particulars of the invention will be explained in more detail with reference to an embodiment shown in the drawing.

There are Shown:

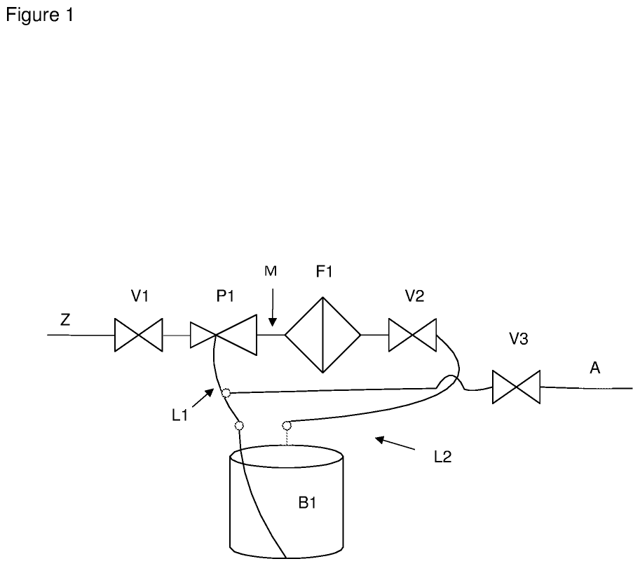

FIG. 1: a schematic view of a first embodiment of the device without a circuit line;

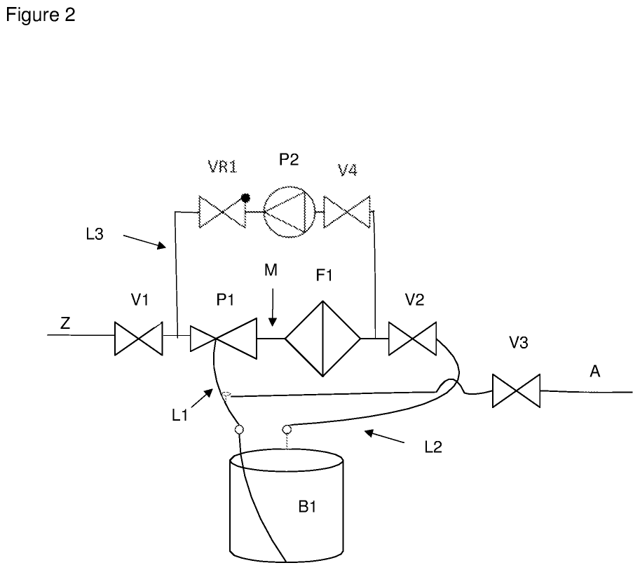

FIG. 2: a schematic view of a second embodiment of the device with a circuit line; and

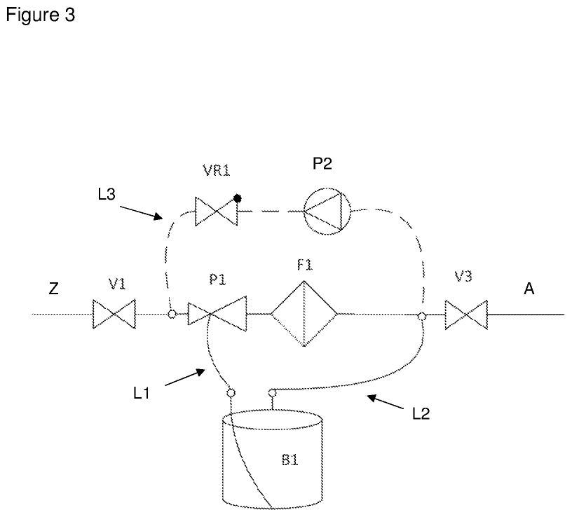

FIG. 3: a schematic view of a third embodiment of the device for degassing the dialysis concentrate.

FIGS. 1 to 3 show different embodiments of the device for preparing dialysis concentrate. Elements that are the same or have the same function are marked by the same reference numerals in the Figures.

In accordance with FIG. 1, the mixing unit comprises the suction unit P1 and the mixing chamber F1.

The suction unit P1 is, on the one hand, connected to a source for water such as RO water or for another solvent via the inlet line Z and is, on the other hand, connected to the suction line L1 that is in turn fluidically connected to the container B1 that contains the raw concentrate material.

The valve V1 is arranged in the inlet line Z upstream of the suction unit P1. The valve V2 is located downstream of the mixing chamber F1. The dialysis concentrate flows via it through the return line L2 to the container B1.

As can further be seen from FIG. 1, the drainage line A is connected to the suction line L1 and the dialysis concentrate is removed from the container B1 by means of it, e.g. through the blood treatment device, and can be used to prepare the ready-to-use dialysis solution. In this respect, a concentrate pump can be used that conveys the liquid dialysis concentrate from the container into a line in which the dialysis concentrate is mixed with water or the like for the purpose of preparing the ready-to-use dialysis solution. A mixing chamber, etc. can be present for this purpose to ensure a sufficient mixing. One or more metering or constricting means can be present in the inflow line into which the concentrate line opens that ensure a specific mixture ratio between the dialysis concentrate and water and thus a specific dilution of the dialysis concentrate.

In the embodiment in accordance with FIG. 1 the sequence for the preparation of the dialysis concentrate is as follows.

The container B1 that contains the raw material (also called the “raw material container” in the following) is first connected to connectors of the device or of the dialysis machine or of the mixing unit by means of the lines L1, L2.

Instead of a dialysis machine, it can also be another preparation device so that the term “dialysis machine” used as part of the invention is to be understood broadly and covers any desired preparation units.

The raw material container B1 has to have a size such that the total volume of the completely mixed dialysis concentrate can be received therein.

The raw material container B1 has two connectors, one for the line 1 and a further one for the line L2.

The raw material container B1 can have flexible or rigid walls and can e.g. be configured as a bag or as a cartridge. It can, for example, be a flexible disposable container, e.g. in the form of one or more bags.

The line L1 is preferably a suction line that extends down to the deepest point of the raw material container B1.

A concentrate that preferably forms an acidic or also a basic dialysis concentrate on its dissolving is located in the container 131. This dialysis concentrate is used to prepare a ready-to-use dialysis solution after a mixing, optionally with a further concentrate and dialysis water.

The water or other solvent, both generally called “dialysis water” as part of the invention, required for the mixing or the dissolving of the raw material is supplied from the inflow connector, i.e. the inlet line Z, to the mixing circuit of the dialysis machine at a defined flow, amount, and pressure. The valve V1 is located in the inflow Z.

The exact metering of the dialysis water can take place e.g. by means of flow sensors or via a volume or mass determination or with constant inflow conditions via a time control of the inflow valve(s) V1 or also via any other desired metering device.

The dialysis water flowing in from the dialysis system or from another source flows via the suction unit P1, that is configured to generate a partial vacuum in the line L1 leading from the container B1 to the mixing circuit. The raw material or the mixture of dialysis water and raw material is thus sucked from the container B1 into the mixing line M by means of the suction unit P1 and the mixture of dialysis water and raw material is then conveyed into the fine mixing chamber F1 that is located in the mixing line M. The suction unit P1 is arranged between the inflow line Z and the mixing line M.

The suction unit P1 is configured such that it can suck in the optionally different forms of the raw material and can then mix them with the dialysis water.

The raw material is dissolved in the fine mixing chamber F1 and the solution then flows via the line L2 back into the container 131. If it is a liquid raw material, a mixing of raw material and dialysis water takes place in the mixing chamber that is also called a fine mixing chamber F1 within the framework of the invention. The fine mixing chamber F1 contains a retention element such as a filter element and acts as self-regulating. If undissolved raw material accumulates at this retention element, the pressure loss in the system rises and the throughflow through the filter element is reduced or is fully prevented. The flow rate via the suction unit and thus a further suction of further raw material from the container is thereby minimized. Even with a small flow, the undissolved raw material is flowed over by fresh solvent and the dissolving is thus optimized. If the raw material has dissolved sufficiently in the fine mixing chamber, the pressure loss via the retention element falls and the flow rate of the solvent through the suction unit increases, which promotes a further suction of raw material through the suction unit into the fine mixing chamber.

The mixing process in the fine mixing chamber F1 is self-regulating by the combination of the embodiment of the suction unit P1 and the fine mixing chamber F1.

If the raw material has dissolved sufficiently in the fine mixing chamber F1, the pressure loss falls and the flow rate through the suction unit P1 increases, which promotes a further suction of raw material through the suction unit P1 into the fine mixing chamber F1. If, in contrast, the raw material has not yet sufficiently dissolved in the fine mixing chamber F1, the pressure loss increases via the suction unit P1 and via the fine mixing chamber F1 so that the flow rate via the suction unit P1 is correspondingly small and a further suction of raw material from the container B1 is thus minimized or completely stopped.

The valve V1 is closed when a specific amount or a specific volume of dialysis water has been supplied.

As can be seen from FIG. 1, the mixing unit comprises the suction unit P1, the fine mixing chamber F1, and the raw material container B1. A valve V2 that can be formed as a check valve VR2 can be located in the mixing line M. The dialysis concentrate flows from it into the container B1.

In the embodiment in accordance with FIG. 1, the mixing of the raw material takes place outside the container B1 and without a further mixing branch.

The mixing circuit with the suction unit P1 and the fine mixing chamber is—as stated above—self-regulated and preferably thus ensures the complete dissolving/mixing of the raw material. Clogging by the raw material is prevented. A sensor system/control unit for monitoring and controlling the mixing process can thus be dispensed with in a preferred embodiment,

In general, the use of exactly one mixing chamber F1 or also the use of a plurality of mixing chambers that are connected in series or in parallel is covered by the invention. In the case of a plurality of mixing chambers, they can be designed as identical or having differently fine mixing elements.

In the event that the raw material does not dissolve completely or not sufficiently, an additional mixing drive branch can be provided such as is shown in FIG. 2. It comprises a pump P2 or another pressure generator that is suitable to generate a fluid flow through the circuit line L3 and a valve V4 and/or a check valve VR1.

As can be seen from FIG. 2, the mixing drive branch is connected in parallel with the suction unit P1 and the mixing chamber F1. The mixing drive branch preferably opens into the mixing line downstream of the valve V1 and upstream of the valve V2 or VR2 such as is shown in FIG. 2.

A valve V4 by means of which the circuit line containing the pump P2 can be blocked can optionally be provided upstream of the pump P1 of the mixing branch.

The solution or suspension is withdrawn from the line section between F1 and V2/VR2 by means of the pressure generator P2, is conveyed by the pump P2, and is then again added into the line section of the mixing line M between V1 and F1. A circuit flow is thus produced via the mixing chamber F1 and the mixing time can be extended independently of the dialysis water supply or of the amount of the supplied dialysis water.

Once the mixing process has ended, the dialysis machine or another device can suck the prepared dialysis concentrate or the dialysis solution or another completed mixing solution from the container B1 through the suction line A, for which purpose the valve V3 is opened and the container B1 is emptied. The line A can generally also be configured as a pressure line.

No collection container in which the mixing procedure is carried out is required for the mixing method. The dissolving/mixing of the raw concentrate material takes place without any previous buffering of dialysis water.

If the dialysis concentrate prepared as describe above is subjected to a degassing, it can take place on the removal with opened valves V2, V3 and a closed valve V1 (and optionally V4) and with a line L1 decoupled from the container B1 or via the mixing chamber F1 with an optional closed valve in the line L1.

The suction line A is connected to the suction line L1 in the embodiments of FIGS. 1 and 2. The completed dialysis concentrate is removed from the container B1 by means of the suction line A.

In the embodiment in accordance with FIG. 3, the line A is connected directly to the mixing chamber F1 and/or directly to the return line L2. The degassing of the dialysis concentrate prepared using this device can take place with an open valve V3 and closed valves V1 and, optionally, V2 and V4 via the mixing chamber F1.

After the dilution or after the preparation of the dialysis concentrate, the system or all the flow paths can be flushed or disinfected, just as is possible in accordance with the prior art with the canister connectors to dialysis machines.

In a further embodiment, a disinfectant could be supplied via the inflow (inlet line Z) or the bag B1 can contain a disinfectant instead of the raw material batch.

The preparation of cleaning, decalcification, and disinfectant solutions or similar is equally conceivable by the proposed device and the method. A cleaning or disinfecting of the connected dialysis system or the like is thus also possible with a solution prepared in the device from concentrated raw material (via the suction or conveying of the solution via the line S).

The method in accordance with the invention can be combined with, for example:

A density measurement for the (automatic) quality control or determination of the progress of the mixing process.

The dialysis machine or a different source can provide temperature controlled dialysis water to optimize and/or accelerate the dissolving process.

The device in accordance with the invention can be combined with a system for online quality monitoring and release.

A combination with a system for quality monitoring and quality controlled optimization of the mixing process with regulation measures (such as the temperature, mixing time, supply of air (optional) or others) is conceivable.

A further possible embodiment of the invention is the integration of the mixing device in accordance with the invention in a system or in a unit that is connected upstream of the dialysis machine and is connected to or integrated with a dialysis water supply so that the preparation of the solution in the container B1 can take place in a preparatory manner.

In a further embodiment, the highly precise metering system of a dialysis machine for the correct metering of the dialysis water for the mixing of the raw material could be connected upstream of the inflow (inlet line Z) of the mixing system and could thus be integrated in the mixing method.

In a preferred embodiment of the invention, the method in accordance with the invention is carried out as follows:
1. The raw material bag or container B1 containing raw material is connected via the lines L1, L2 to the corresponding connectors of the dialysis machine.
2. Dialysis water is transferred from the inflow connector (inlet line Z) with the blocking device V1 at a defined flow, amount, and pressure to the mixing circuit.
3. The dialysis water provided by the dialysis system or from another source at a defined flow, amount, and pressure flows over the suction unit P1 and thus sucks the raw material or the raw material/dialysis water mixture from the container B1 such as a bag or a cartridge and transports the raw material/dialysis water mixture into the fine mixing chamber F1. The raw material is dissolved in the dialysis water or mixed with it therein and the completed solution flows back in the form of the dialysis concentrate via the line L2 into the container B1.
4. After providing the defined amount of dialysis water, the blocking valve V1 is closed.
5. If a raw material should be used that does not fully dissolve during the dialysis water inflow phase, the raw material can be completely dissolved with the aid of the optional mixing drive branch in accordance with FIG. 2 or 3.
6. After ending the mixing process, the dialysis machine can suck the dialysis concentrate or the dialysis solution or a different completed mixing solution from the bag B1 by means of the line 2 and the open valve V3.
7. A density measurement or similar methods for (automatic) quality control or determining the progress of the mixing process can optionally be used.
8. Temperature controlled dialysis water that could e.g. be provided by the dialysis machine can be used to optimize and/or accelerate the dissolving process.
9. After a successful end of the dialysis treatment, the bag B1 can be residually emptied by the machine and can subsequently be decupled and disposed of. Alternatively, the bag can also first be decoupled and then manually residually emptied and disposed of.
10. Finally, the dialysis machine carries out a cleaning or disinfecting process with feedback connectors, analogously to the cleaning process for the canister connectors.
11. Alternatively to the feedback of the connectors, a cleaning or disinfecting process can be carried out by connecting a bag with cleaning/disinfectant solution.
12. The cleaning/disinfectant solution used could here also be prepared by mixing on the basis of a concentrate analogously to the preparation of the dialysis concentrate by the device in accordance with the invention, with the preparation then being an integral component of the cleaning/disinfectant process.

The integration of the mixing process of the raw material from a raw material batch (such as from a bag) e.g. in dialysis machines is key. A second mixing circuit with a mixing tank is not necessary here. The design of the suction unit P1 and of the fine mixing unit F1 for the automatic, self-regulating, safe dissolving or preparation of the dialysis concentrate or of the cleaning or disinfection solution and the combination of container or bag with raw material concentrate, method, and device is furthermore advantageous for a safe self-regulating dissolving.

A collection container or active pump device is not absolutely necessary for the mixing method in accordance with the present invention.

Advantages of the present invention in a preferred embodiment are:
small investment costs
high flexibility for the treatment with different dialysis concentrates
bag with concentrate raw material is lighter than canisters with dialysis concentrate or a batch with completed solution for dialysis treatment
good storage capability and durability
smaller waste production
smaller transport effort
smaller operating costs in comparison with canisters having dialysis concentrate or completed solution for a dialysis treatment.

1. A device for preparing a dialysis concentrate or another fluid, wherein the device includes an inflow (Z) for a solvent for dissolving or mixing with a raw material that is located in a container (B1) and that can be dissolved in the solvent or is miscible with it, wherein the device has connection means for connection to the container, characterized in that the device has a mixing unit that is self-regulating and comprises a mixing line (M) and a suction unit (P1) arranged in the mixing line (M), and a mixing chamber (F1), with the suction unit (P1) being able to be brought into fluid communication with the inflow (Z) for the solvent and, by means of a suction line (L1), with the container (B1) connectable to the device, and with a return line (L2) downstream of the mixing chamber (F1) leading back to the container (B1) connectable to the device.

2. A device in accordance with claim 1, characterized in that a valve (V1) is arranged in the mixing line (M) upstream of the suction unit (P1); and/or in that a valve (V2) is arranged in the mixing line (M) downstream of the mixing chamber (F1).

3. A device in accordance with claim 1, characterized in that the mixing unit does not have any mixing tank except for the mixing chamber (F1).

4. A device in accordance with claim 1, characterized in that a drainage line/A) is provided for draining off the completed dialysis concentrate or the other fluid, with the drainage line (A) being fluidically connectable to the container (B1) or being connected to the suction line (L1) or to the mixing chamber (F1).

5. A device in accordance with claim 1, characterized in that a circuit line (L3) is provided that is arranged in parallel with the suction unit (P1) and/or the mixing chamber (F1) and that opens into the mixing line (M) upstream of the suction unit (P1) or of the mixing chamber (F1) and downstream of the mixing chamber (F1); and/or in that the mixing chamber (F1) is a filter chamber, preferably a fine mixing filter chamber (F1).

6. A device in accordance with claim 5, characterized in that a pump (P1) and preferably at least one valve (VR1, V4) is arranged in the circuit line (L3).

7. A device in accordance with claim 5, characterized in that a sensor is provided that is configured to detect a parameter value of the fluid in or downstream of the mixing chamber (F1) characteristic for the progress of the dissolving or mixing of the raw material in the or with the solvent, with a control unit being present that is connected to the sensor and that is configured to open or to close the circuit line (L3) in dependence on the parameter value.

8. A device in accordance with claim 1, characterized in that the suction unit is a Venturi nozzle.

9. An arrangement comprising a device in accordance with claim 1 and a container (B1) that contains the raw material and that is in fluid communication with the device such that raw material from the container (B1) can be conducted to the suction unit (P1) and solution can be conducted from the mixing line (M) into the container (B1).

10. A blood treatment device, in particular a dialysis machine, having a device or an arrangement for preparing a dialysis concentrate or another fluid in accordance with claim 1.

11. A blood treatment device in accordance with claim 10, characterized in that the blood treatment device has a water inflow line and a concentrate line branching off therefrom that is in fluid communication with the dialysis concentrate located in the container (B1), with a mixing region being provided in which the dialysis concentrate conveyed out of the container (B1) is mixed for the purpose of preparing a ready-to-use dialysis solution with the water from the water inflow line.

12. A method of preparing a dialysis concentrate or another fluid, characterized in that the method is carried out using an arrangement in accordance with claim 9.

13. A method in accordance with claim 12, characterized in that the preparation of the dialysis concentrate or of the other fluid is carried out without a circuit flow of the raw material taking place via the container (B1).

14. A method in accordance with claim 12, characterized in that the circuit line (L3) is flowed through when the solution/mixture of the raw material in the solvent is not yet complete after metering in a specific solvent amount.

15. A method in accordance with claim 12, characterized in that the solution or a first partial flow thereof is branched off from the mixing line downstream of the suction unit (P1) and is again conducted via the suction unit (P1) to convey still undissolved or unmixed substances from the container (B1), with provision preferably being made that a second partial flow is supplied from the mixing line (M) to the container (B1).

16. A method in accordance with claim 12, characterized in that a degassing of the dialysis concentrate or of the other fluid in the mixing chamber (K1) takes place.

17. Use of a device or arrangement in accordance with claim 1, characterized in that the device or arrangement for preparing a dialysis concentrate or for preparing another fluid, in particular a cleaning, sterilizing, or disinfecting fluid, in particular for blood treatment devices, preferably for dialysis machines, is used.
